(19) United States
(12) Patent Application Publication
(10) Pub. No.: US 2019/0015311 A1
(43) Pub. Date: Jan. 17, 2019

(54) OIL-BASED EYELASH COSMETIC AND COSMETIC METHOD

(71) Applicant: JO Cosmetics Co., Ltd., Tokyo (JP)

(72) Inventors: Mizuyo INOUE, Tokyo (JP); Yumi TOMIYAMA, Tokyo (JP); Hazuki UCHIDA, Tokyo (JP); Akihiro NAKANO, Tokyo (JP); Satoshi HARAMIZU, Tokyo (JP)

(73) Assignee: JO Cosmetics Co., Ltd., Tokyo (JP)

(21) Appl. No.: 16/070,020

(22) PCT Filed: Jan. 13, 2017
(86) PCT No.: PCT/JP2017/000906

§ 371 Date: Jul. 13, 2018

(30) Foreign Application Priority Data
Jan. 16, 2016 (JP) 2016-006698

(51) Int. Cl. A61K 8/58; A61Q 1/10; A61K 8/31; A61K 8/73; A61K 8/60; A61K 8/37; A61K 8/891; A61K 8/49; A61K 8/19; A61K 8/34

(52) U.S. Cl. CPC A61K 8/585; A61Q 1/10; A61K 8/31; A61K 8/732; A61K 8/60; A61K 8/345; A61K 8/37; A61K 8/891; A61K 8/498; A61K 8/19; A61K 8/375

(57) ABSTRACT

An oil-based eyelash cosmetic including 3 to 40% by mass of an oil-soluble film forming agent (A), 0.1 to 10% by mass of a nonionic surfactant having an HLB value of 7 to 10 (B), 20 to 70% by mass of a volatile oil (C) and 0.5 to 30% by mass % of a nonvolatile oil (D), wherein the ratio of the component (A) and the component (D) [(A)/(D) (mass ratio)] is 0.7/1 to 5.8/1. When a solid oil having a melting point of 50° C. or higher is contained, the amount thereof is 10% by mass or less. The cosmetic exhibits excellent removability by warm water as well as excellent curling effect and long-lasting performance of cosmetic effect such as water resistance peculiar to conventional oil-based eyelash cosmetics.

### DETAILED DESCRIPTION OF THE INVENTION

### Technological Field

The present invention relates to an oil-based eyelash cosmetic and a cosmetic method for making up eyelashes using the same, and more specifically, relates to an oil-based eyelash cosmetic excellent in removability by washing with warm water and a cosmetic method for making up the eyelashes with the same in which the makeup can be readily removed by washing with warm water.

### Background Technology

An eyelash cosmetic, a representative of which is mascara, has a makeup effect to make eyes striking by curling eyelashes upwards or making the eyelashes thick and long. Conventionally, various types of eyelash cosmetics such as oil-based type, water-in-oil emulsion type, oil-in-water emulsion type and aqueous type have been known. In recent years, a mascara of the oil-in-water emulsion type that can be readily removed by washing with warm water without using a special remover has become popular.

However, the mascara of the oil-in-water emulsion type has a problem that curling imparted to the eyelashes is hard to be maintained while the mascara is excellent in removability (cleansing property), that is, the mascara can be readily removed. This is because the eyelashes which are physically curled by using a tool such as an eyelash curler lose the curling effect due to contact with moisture.

On the other hand, the oil-based eyelash cosmetic has an excellent curl-retaining effect as compared with the eyelash cosmetic of the emulsion type such as the oil-in-water type or the water-in-oil type, and also is excellent in long-lasting performance of cosmetic effects such as water resistance (refer to Patent Document 1, paragraph 0009). However, the oil-based eyelash cosmetic has a disadvantage that a special remover is necessary for removing makeup. Hence, studies to improve the removability of the makeup have been intensively conducted. As a solution to the problem, an oil-based eyelash cosmetic wherein a sufficient removability for removing makeup with warm water is imparted by blending an oil-soluble resin, a fatty acid soap, a nonionic surfactant having an HLB value of 12 or more and a volatile oil is proposed (refer to Patent Document 2). However, according to experiments by the inventors of the present invention, oil-based eyelash cosmetics prepared by using a surfactant such as a fatty acid soap or nonionic surfactant having a high HLB value did not yet exhibit sufficient removability, and also were insufficient in water resistance against sweat and tears.

Further, Patent Document 3 proposes an eye-makeup cosmetic containing an oily gelling agent, a high viscosity hydrocarbon oil, a polar oil and a volatile oil, and describes that the cosmetic is excellent in long-lasting performance of cosmetic effects, smoothness of a makeup film, gloss upon application and cleansing property (refer to claim 1 and paragraph 0007). Patent Document 3 also describes that incorporation of a surfactant to the cosmetic, in particular, incorporation of a nonionic surfactant having an HLB value of less than 10 together with a nonionic surfactant having an HLB value of 10 or more improves the cleansing property (refer to paragraphs 0023 to 0025), and discloses specific formulations containing polyethylene glycol monolaurate having an HLB value of 14 together with isostearyl glyceryl ether having an HLB value of 5.3 in its examples (refer to Examples 1 to 2 and 4 to 6, Table 1). In these examples, the makeup cosmetics are evaluated as being excellent in cleansing property. However, the evaluation of cleansing property in these examples is performed according to the method in which the makeup is washed with a soap after making an oil-based cleansing agent blend to the eyelashes. Patent Document 3 is silent about the removability of a cosmetic upon cleansing the makeup with warm water alone (refer to paragraph 0039).

In addition, Patent Document 4 discloses a preservative fungicide containing a tyrosol derivative having a specific chemical structure (refer to claim 1) and a cosmetic containing the tyrosol derivative (see claim 3). It also discloses, in examples 30 to 32, mascaras of the water-in-oil type which contain an oil-soluble film forming agent, a nonionic surfactant, a volatile oil, a nonvolatile oil and an oil gelling agent, and further contain 10% by mass of beeswax and carnauba wax 5% by mass of carnauba wax relative to the whole composition wherein the total content of both waxes is 15% by mass. The technical feature of the invention described in Patent Document 4 is to provide a novel antiseptic bactericidal agent, hence, the document merely mentions a mascara of the water-in-oil type as an example of applications using the antiseptic bactericidal agent. Therefore, the document discloses nothing about the characteristics of the mascara.

Further, Patent Document 5 discloses an oil-based mascara that contains 6 to 35% by mass of a wax having a melting point of 55 to 70° C. (A), 0.1 to 10% by mass of a surfactant having an HLB value of 5 to 10 (B), 10 to 90% by mass of a volatile hydrocarbon (C) and, as an optional component, an oil-soluble film-forming agent and a nonvolatile oil, wherein the content of the component (A) in the whole wax is 85% by mass or more (refer to claim 1). In Examples 1 to 3, oil-based eyelash cosmetics prepared by blending a wax, a nonionic surfactant, a volatile oil, an oil soluble film forming agent, a nonvolatile oil and an oil gelling agent are described. The oil-based eyelash cosmetics have drawbacks that luster of eyelashes is difficult to be obtained since the wax having a melting point of 55 to 70° C. is contained in a range of 6 to 35% by mass, and removability by washing with warm water is insufficient since the content of the oil-soluble film forming agent is larger than the content of the nonvolatile oil.

### PRIOR ART DOCUMENT

### Patent Document

Patent Document 1: Japanese Unexamined Patent Publication No. 2006-306829
Patent Document 2: Japanese Unexamined Patent Publication No. 2006-265216
Patent Document 3: Japanese Unexamined Patent Publication No. 2008-115120
Patent Document 4: Japanese Unexamined Patent Publication No. 2007-039340
Patent Document 5: Japanese Unexamined Patent Publication No. 2012-1403384

### DISCLOSURE OF THE INVENTION

### Problems to be Solved by the Invention

The present invention was completed under the aforementioned background, and it is therefore an object of the present invention to provide an oil-based eyelash cosmetic capable of imparting removability that is sufficient for removing makeup by washing with warm water alone without use of a special remover in the same manner as an eyelash cosmetic of the oil-in-water type while maintaining excellent curl effect and water resistance peculiar to conventional oil-based eyelash cosmetics.

### Means Used to Solve the Problem

As a result of intensive studies to solve the above-mentioned problems, the present inventors have found that use of an oil-soluble resin and a nonvolatile liquid oil at a specific ratio and use of a nonionic surfactant having a specific HLB value as a surfactant result in an oil-based eyelash cosmetic that is excellent in curling effect, and makeup obtained by using the oil-based eyelash cosmetic can be readily removed by cleansing with warm water despite exhibiting good water resistance. The present invention was completed based on the above knowledge.

Thus, the present invention provides an oil-based eyelash cosmetic comprising 3 to 40% by mass of an oil-soluble film forming agent (A), 0.1 to 10% by mass of a nonionic surfactant having an HLB value of 7 to 10 (B), 20 to 70% by mass of a volatile oil (C) and 0.5 to 30% by mass of a nonvolatile oil (D), wherein the ratio by mass of the component (A) to the component (D) [(A)/(D)] is 0.7/1 to 5.8/1, and when a solid oil having a melting point of 50° C. or higher is contained, its content is 10% or less by mass.

### Effect of the Invention

The oil-based eyelash cosmetic of the present invention is excellent in removability by washing with warm water in addition to excellent curling effect and long-lasting performance of cosmetic effect such as water resistance peculiar to the conventional oil-based eyelash cosmetics. Further, according to the cosmetic method of the present invention, makeup can be readily removed by cleansing with warm water without using a special remover.

### EMBODIMENTS FOR CARRYING OUT THE INVENTION

The oil-based eyelash cosmetic of the present invention contains an oil-soluble film forming agent (A), a nonionic surfactant having an HLB value of 7 to 10 (B), a volatile oil (C) and a nonvolatile oil (D) as essential components.

The oil-soluble film-forming agent used as the component (A) is not particularly limited as long as it dissolves in an oily component to form a film, and examples thereof include silicone resins such as trimethylsiloxysilicate, partially crosslinked organopolysiloxane, trimethylsiloxysilyl propyl carbamic acid, fluorine-modified silicone, acrylic modified silicone, silicone dendrimer-modified resin compound; rosin acid resins such as pentaerythrityl rosinate, glyceryl rosinate; candelilla resin, polyvinyl acetate type resin, polyvinyl isobutyl ether, polyisobutylene and the like. Here, candelilla resin means a resin component obtained by separating and extracting candelilla wax with an organic solvent, and has a resin content of preferably 65% or more, more preferably 85% or more.

Of these, trimethylsiloxysilicate, rosin acid resins and candelilla resin provide better long-lasting performance of cosmetic effect and curling effect. Examples of commercially available products include X-21-5595, KF-7312J, KF-7312F (Shin-Etsu Chemical Co., Ltd.) which is a solution of trimethylsiloxysilicate, PINECRYSTAL KE-311 which is glyceryl hydrogenated rosinate (Harima Chemicals, Inc.), candelilla resin E-1 (Japan Natural Products Co., Ltd.). The oil-soluble film-forming agent can be used solely or in combination as appropriate.

The content of the component (A) is usually 3% to 40% by mass, preferably 5% to 35% by mass, and more preferably 7% to 30% by mass with respect to the whole cosmetic. When the content of the component (A) is excessively small, luster of eyelashes, curling effect and water resistance become insufficient. Conversely, when it is excessively large, removability by warm water tends to decrease and application to the eyelashes becomes difficult.

### (Surfactant)

In the present invention, a nonionic surfactant is used as the component (B). The nonionic surfactant used has an HLB value in the range of 7 to 10, preferably 8 to 9. When the nonionic surfactant has an HLB value of less than 7, removability of makeup by warm water cannot be improved. Even if an anionic surfactant or a cationic surfactant is used in place of the nonionic surfactant, an eyelash cosmetic having excellent removability by warm water cannot be obtained.

Examples of the nonionic surfactant used as the component (B) include polyglycerol fatty acid esters such as polyglyceryl-6 stearate, polyglyceryl-10 tristearate and polyglyceryl-6 oleate; polyoxyethylene hydrogenated castor oil such as PEG-10 hydrogenated castor oil and PEG-20 hydrogenated castor oil; polyoxyethylene fatty acid esters such as PEG-5 stearate and PEG-6 isostearate; polyoxyethylene alkyl ethers such as ceteth-4, oleth-6 and steareth-6; fatty acid polyoxyethylene alkyl ethers such as steareth-12 stearate and laureth-10 isostearate; polyoxyethylene fatty acid glyceryl such as PEG-6 glyceryl isostearate, PEG-20 glyceryl triisostearate and PEG-20 glyceryl tristearate; fatty acid polyoxyethylene hydrogenated castor oil such as PEG-20 hydrogenated castor oil isostearate, PEG-40 hydrogenated castor oil triisostearate, polyoxyethylene sorbitan fatty acid ester, polyoxyethylene polyoxypropylene copolymer, ether of polyoxyethylene polyoxypropylene copolymer and long-chain alcohol, ether of polybutylene glycol polyglycerin copolymer and long-chain alcohol, and the like. Of these, polyoxyethylene fatty acid ester, polyoxyethylene fatty acid glyceryl and fatty acid polyoxyethylene alkyl ether are preferably used.

When the nonionic surfactant contains a fatty acid residue in its molecule, it is preferably a residue of a higher fatty acid having 10 to 22 carbon atoms such as myristic acid, palmitic acid, stearic acid, isostearic acid and oleic acid. Examples of commercial products suitably used as the component (B) include Emalex GWS-320 (PEG-20 glyceryl tristearate; Nihon Emulsion Co., Ltd.; HLB 8), UNIOX GT-20IS (PEG-20 glyceryl isostearate; NOF Corporation; HLB 8), EMALEX PEIS-6EX (PEG-6 isostearate; Nihon Emulsion Co., Ltd.; HLB 9), and EMALEX SWS-12 (steareth-12 stearate; Nihon Emulsion Co., Ltd.; HLB 8).

The component (B) may be used alone or in combination of two or more. The content of the component (B) used is 0.1 to 10% by mass, preferably 0.5 to 8% by mass, and more preferably 1 to 6% by mass in the whole composition. When the content is excessively small, removability by warm water decreases, whereas when it is excessively large, water resistance decreases.

In the present invention, in addition to the component (B), a nonionic surfactant having an HLB value of more than 10, an anionic surfactant, a cationic surfactant or an amphoteric surfactant can be appropriately used in combination. Since these surfactants sometimes lower water resistance of the cosmetic, in the case of containing these surfactants, the content thereof is preferably controlled to 2% by mass or less, more preferably 0.5% by mass or less, further more preferably 0.1% by mass or less with respect to the whole composition. In particular, it is preferred that the content is controlled to 0.05% by mass or less.

### (Volatile Oil)

The volatile oil used as the component (C) in the present invention is not limited as long as it is used in conventional cosmetics. The volatile oil usually has a boiling point at normal pressure of 60° C. to 260° C., preferably 100° C. to 220° C. Examples thereof include hydrocarbon oils such as isododecane, isohexadecane and saturated isoparaffin-type hydrocarbon oils containing a compound having 8 to 16 carbon atoms as a main component (e.g. hydrogenated polyisobutene); and volatile silicone oils. Examples of commercially available products include Marcasol R available from Maruzen Petrochemical Co., Ltd., IP Solvent 1620 and IP Solvent 2028, both of them are available from Idemitsu Kosan Co., Ltd., and the like. Of these, in view of storage stability and volatilization rate, isododecane and hydrogenated polyisobutene containing a compound having 8 to 16 carbon atoms as a main component are preferably used.

The content of the component (C) used is 20 to 70% by mass, preferably 30 to 65% by mass, more preferably 35 to 60% by mass with respect to the whole composition. If the content is excessively small, curling effect and water resistance are lowered. Conversely, if the content is excessively large, an amount of residual components on the eyelashes is reduced, thereby volume effect and curling effect decrease.

### (Nonvolatile Oil)

The nonvolatile oil used as the component (D) in the present invention is a liquid or pasty oil having a higher boiling point than the component (C) at ambient temperature. Such nonvolatile oil is not particularly limited as long as it is used in conventional cosmetics, and may be any animal oil, vegetable oil or synthetic oil. Examples of the nonvolatile oil include esters such as triethylhexanoin, diisostearyl malate, polyglyceryl-2 isostearate, diglyceryl triisostearate, decaglyceryl decaisostearate, oligomeric esters of dimer acid and dimer diol, pentaerythrityl tetraisostearate, diglyceryl tetraisostearate, cetyl isooctanoate, isopropyl myristate, isopropyl palmitate, octyldodecyl myristate, neopentyl glycol dioctanoate, cholesterol fatty acid ester and jojoba oil; hydrocarbons such as polybutene, polyisobutylene, heavy liquid isoparaffin, liquid paraffin, α-olefin oligomer, squalene, petrolatum and polyisobutylene; fats and oils such as olive oil, castor oil, mink oil and macadamia nut oil; fatty acids such as isostearic acid and oleic acid; higher alcohols such as oleyl alcohol and isostearyl alcohol; silicone oils such as dimethyl polysiloxane having a low polymerization degree, dimethyl polysiloxane having a high polymerization degree, methylphenyl polysiloxane, cross-linked organopolysiloxane and fluorine-modified polysiloxane; fluorine type oil such as perfluoropolyether; lanolin derivatives such as lanolin, lanolin acetate, lanolin fatty acid isopropyl and lanolin alcohol; and the like. The nonvolatile oil of the component (D) may be a nonionic surfactant having an HLB value of less than 7 which is a liquid or pasty oil at ambient temperature. The nonvolatile oil (D) may be used alone, or in combination of two or more.

The content of the component (D) used is 0.5 to 30% by mass, preferably 1 to 20% by mass, more preferably 2 to 15%, by mass with respect to the whole composition. Incorporation of the component (D) within this range enables a cosmetic to have excellent curling effect, water resistance and removability of makeup by warm water.

In the present invention, the ratio by mass of the component (A) to the component (D), which is represented by (A)/(D), is 0.7/1 to 5.8/1, preferably 1/1 to 5.8/1, more preferably 1.5/1 to 5.5/1. If the ratio of the component (A) is excessively large, removability by warm water is lowered. Conversely, if the ratio is excessively small, water resistance is lowered.

In the cosmetic of the present invention, an oily gelling agent can be added as the component (E) in addition to the components (A) to (D). Incorporation of the oily gelling agent enables a cosmetic to have an appropriate viscosity for its application to the eyelashes, and to readily maintain a uniform and stable state. Examples of the oily gelling agent include dextrin fatty acid ester, inulin fatty acid ester, sucrose fatty acid ester, starch fatty acid ester, organically modified clay mineral, metal soap, 12-hydroxystearic acid and the like.

The dextrin fatty acid ester is an ester of dextrin or reduced dextrin and a higher fatty acid, and examples thereof include dextrin palmitate and dextrin palmitate/2-ethylhexanoate. Examples of commercially available products include dextrin palmitate such as Rheopearl KL 2 and Reopearl TL 2; and dextrin palmitate/2-ethylhexanoate such as Rheopearl TT 2, any of which is available from Chiba Flour Milling Co., Ltd. Examples of the sucrose fatty acid esters include sucrose stearate and sucrose acetate stearate, and examples thereof include Sugar Wax S-10 E, DK Ester S-160, and Sugar Wax A-10 E, any of which is available from DKS Co. Ltd.

Examples of the organically modified clay mineral include those obtained by substituting a convertible cation interposed between crystal layers of a clay mineral such as montmorillonite, saponite, hectorite and bentonite with an organic polar compound or an organic cation. Specific examples include dimethyl distearyl ammonium hectorite (i.e. disteardimonium hectorite), dimethyl distearyl ammonium bentonite (i.e. quaternium-18 bentonite), dioctadecyl dimethyl ammonium salt-modified montmorillonite, octadecyl dimethyl benzyl ammonium salt-modified montmorillonite, dihexadecyldimethyl ammonium salt-modified montmorillonite. Examples of commercially available products include Bentone 38 V (disteardimonium hectorite), Bentone 34 (quaternium-18 bentonite), Bentone 27 (benzyl dimethyl distearyl ammonium hectorite), any of which is available from Elementis Corporation. Examples of the metal soap include aluminum stearate and calcium stearate. The component (E) can be used alone or in combination of two or more.

The content of the component (E) used is 1 to 30% by mass, preferably 3 to 25% by mass, more preferably 5 to 20% by mass with respect to the whole composition. Incorporation of the component (E) enables a cosmetic to readily have an appropriate viscosity for applying a sufficient amount of the cosmetic to the eyelashes using a brush, and also enables it to have improved stability.

In the cosmetic of the present invention, a coloring material can be blended as the component (F). The coloring material is not particularly limited by the shape, particle diameter, and particle structure as long as it is commonly used in the field of cosmetics. Examples of the shape include a spherical shape, a plate shape, and a needle shape. Examples of the particle size include an aerosol, a fine particle and a pigment grade. The particle structure may be porous or nonporous.

Examples of the colorant (F) include inorganic powders, glittering powders, organic powders, pigment powders, metal powders, composite powders, and the like. More specifically, there can be mentioned inorganic pigments such as talc, mica, kaolin, calcium carbonate, silica, zinc oxide, titanium dioxide, red iron oxide, yellow iron oxide, black iron oxide, ultramarine blue, iron blue, carbon black, low order titanium oxide, cobalt violet, chromium oxide, chromium hydroxide, cobalt titanate, bismuth oxychloride and titanium-mica-based pearl pigment; resin powders of organic polymer such as polyamide resin, polyethylene resin, polyacryl resin, polyester resin, fluorine resin, cellulose resin, polystyrene resin, copolymer resin such as styrene-acryl copolymer resin, polypropylene resin, silicone resin, urethane resin; organic pigment such as Red No. 201, Red No. 202, Red No. 204, Red No. 205, Red No. 220, Red No. 226, Red No. 228, Red No. 405, Orange No. 203, Yellow No. 205, Yellow No. 4, Yellow No. 5, Blue No. 1, Blue No. 404, Green No. 3, zirconium lake, barium lake or aluminum lake; natural coloring materials such as chlorophyll and (3-carotene; dyes and the like. These coloring materials may be used alone or in combination of two or more. Of these, black coloring materials, particularly black iron oxide and carbon black are preferably used.

The content of the colorant (F) can be appropriately selected. It is preferably 0.1 to 20% by mass, more preferably 0.5 to 15% by mass with respect to the whole composition. When the content of the coloring material is excessively large, adhesion to the eyelashes tends to be lowered. Conversely, when it is excessively small, coloring effect sometimes becomes insufficient.

The oil-based eyelash cosmetic composition of the present invention may contain a small amount of water as the component (E). The content of water is preferably 5% by mass or less, more preferably 0.1 to 3% by mass with respect to the whole cosmetic. When the content is within the range, the nonionic surfactant (B) and water form a solubilized state, that is, a state where oily components are present as a dispersion medium and the component (B) and water form reversed micelles. As a result, the nonionic surfactant of the component (B) is readily dissolved in the oil phase formed by the component (A) and the component (C), and thereby, separation and precipitation of the component (B) hardly occur even in storage for a long period of time, and storage stability is improved.

In addition to the above components, the oil-based eyelash cosmetic of the present invention may contain other components which are used in conventional cosmetics, for example, additives such as solid oils, polyhydric alcohols, lower alcohols, ultraviolet absorbers, ultraviolet scattering agents, humectants, fragrances, antioxidants, preservatives, sequestering agents, defoaming agents, fibers, dyes, various extracts as long as they do not substantially impair the effect of the present invention. The solid oil to be used has a melting point of 50° C. or more, and may be animal wax, vegetable wax, mineral wax, silicone wax, or synthetic wax. When the solid oil is contained, its content is preferably 10% by mass or less, more preferably 5% by mass or less with respect to the whole composition since incorporation of a large amount of the solid oil decreases luster of the eyelashes upon application to the eyelashes.

The oil-based eyelash cosmetic of the present invention can be prepared in accordance with conventional methods. For example, it can be prepared by combining all of the raw materials and heating the resultant mixture, and cooling the mixture with uniform stirring. Also the oil-based eyelash cosmetic can be prepared by preliminarily mixing the coloring materials with a part of the oil component and/or the surfactant component, subsequently combining the resultant mixture with the remaining components, and heating the mixture.

The oil-based eyelash cosmetic can be used as a mascara, a mascara makeup base, a mascara top coat, an eyelash essence and the like. The form of the cosmetic may be appropriately selected in accordance with its application. For example, the form can be creamy, liquid or the like. The oil-based eyelashes cosmetic of the present invention can be applied to the eyelashes using appropriate tools such as brushes, resin molding applicators made of plastics, applicators made of metals and the like.

In the case of making up the eyelashes using the oil-based eyelash cosmetic of the present invention, the makeup can be readily removed by cleansing with warm water of about 35° C. to 45° C. without using a remover which is usually used for removing makeup of oil-based eyelash cosmetics.

### EXAMPLES

Hereinafter, the present invention will be further described with reference to Examples and Comparative Examples, but the present invention is not limited by these Examples. The content of each ingredient in formulations in the following description is expressed in % by mass with respect to the whole composition unless otherwise specified.

The evaluation methods of the oil-based eyelash cosmetic in the following Examples and Comparative Examples are as follows.

### (Evaluation Methods a to e)

As to the following evaluation items of a to e, ten evaluators applied each sample to their own eyelashes, and scored at seven levels of from 0 to 6 based on the evaluation criteria shown in (1) below. Using the total score of 10 evaluators, properties as an oil-based eyelash cosmetic were evaluated according to the 4-step criterion shown in (2) below. The water resistance of the evaluation item c and the curl-retaining effect of the evaluation item d were evaluated based on the degree of bleeding of the cosmetic at 6 hours after application, and the curl-retaining effect of the evaluation item d was evaluated based on the curling state at 6 hours after application. Further, the removability I by warm water with 40° C. of the evaluation item e was evaluated by immersing a commercial cotton into warm water (tap water) of 40° C., sandwiching the eyelashes applied a sample by the cotton for 30 seconds, wiping off the sample, and visually observing the residual amount of the sample on the eyelashes.

### (Evaluation Item)

a. Volume Effect
b. Curling effect
c. Water resistance (normal temperature) I
d. Curl-retaining effect
e. Removability by warm water of 40° C. I

### (1) Evaluation Criteria

### (Rating): (Evaluation)

6: Very good

### 5: Good

4: Slightly good

### 3: Normal

### 2: Somewhat bad

### 1: Bad

### 0: Very bad

### (2) Four-Grade Criteria

### (Evaluation): (Total Score of Evaluation Score)

⊚: Total point is 46 to 60
◯: Total points is 31 to 45
Δ: Total points is 16 to 30
x: Total points is 0 to 15

### (Evaluation Methods f to g)

As to the removability and water resistance by warm water of 40° C., in addition to the evaluation of the evaluation items e or c, the evaluations shown in the following f or g were carried out, respectively.

Evaluation method f: Water resistance at normal temperature II

A commercial cotton cloth was immersed into tap water at 20° C. A false eyelash to which 0.005 g of each sample was applied was sandwiched by the cotton cloth for 30 seconds. Then the false eyelash was rubbed 30 times by the cotton cloth from its base toward its tip end. The state of sample transferred to the cotton was observed and evaluated according to the 4-grade criterion shown in (3) below. The transfer amount of the sample to the cotton cloth was estimated by visual observation in terms of criteria that a transfer amount obtained when the same test was performed using a cotton cloth containing a sufficient amount of cleansing lotion (product name: Uruochimizu cleansing lotion, Mandom Corporation) is 100%, and a transfer amount when no transfer was observed is 0%.

### Evaluation Method g: Removability by Warm Water of 40° C. II

A commercial cotton cloth was immersed into tap water at 40° C. A false eyelash to which 0.005 g of each sample was applied was sandwiched by the cotton cloth for 30 seconds. Then the false eyelash was rubbed 30 times by the cotton cloth from its base toward its tip end. The transfer amount of the sample to the cotton cloth was estimated in the same manner as (3) described above, and evaluated according to the 4-grade criterion shown in (4) below.

### (3) Four-Grade Criterion (Water Resistance Test)

### (Evaluation): (Evaluation Criteria)

⊚: 10% or less
∘: more than 10% to 50% or less
Δ: more than 50% to 90% or less
x: more than 90%

### (4) Four-Grade Criteria (Removability Test by Warm Water)

### (Evaluation): (Evaluation Criteria)

⊚: more than 90%
∘: more than 50% to 90% or less
Δ: more than 10% to 50%
x: 10% or less

### Examples 1 to 5 and Comparative Examples 1 to 4

### <Oil-Based Mascara>

Mascara of the formulation shown in Table 1 was prepared according to the following production procedure, and as to the cosmetic effect to make the eyelashes thick (volume effect and curling effect), its retention (water resistance and curling effect), and the easiness on removal by washing with warm water, sensory evaluation were carried out according to the following method. The results are also shown in Table 1.

### (Production Procedure)

(1) Ingredients (A) to (D) shown in Table 1 were heated to about 110° C. and homogeneously mixed.
(2) Ingredients (E) to (H) shown in Table 1 were added to the mixed solution obtained in the above (1) and homogeneously mixed.
(3) The mixture prepared in (2) above was filled into a container to form an oil-based mascara.

TABLE 1

| | | | Exampe | | | | | Comparative Example | | | |
|---|---|---|---|---|---|---|---|---|---|---|---|
| Phase | Component | | 1 | 2 | 3 | 4 | 5 | 1 | 2 | 3 | 4 |
| A | (C) | Isododecane (*1) | Balance | Balance | Balance | Balance | Balance | Balance | Balance | Balance | Balance |
| | (E) | Dextrin palmitate (*2) | 15.5 | 15.5 | 15.5 | 15.5 | 15.5 | 15.5 | 15.5 | 15.5 | 15.5 |
| | (A) | Glyceryl hydrogenated rosinate (*3) | 5.0 | 5.0 | 5.0 | 5.0 | 5.0 | 5.0 | 5.0 | 5.0 | 5.0 |
| | (E) | Sucrose acetate stearate (*4) | 1.0 | 1.0 | 1.0 | 1.0 | 1.0 | 1.0 | 1.0 | 1.0 | 1.0 |
| | (D) | Polyglyceryl-2 isostearate (*5) | 2.0 | 2.0 | 2.0 | 2.0 | 2.0 | 2.0 | 2.0 | 2.0 | — |
| B | (D) | Triethylhexanoin (*6) | 2.0 | 8.0 | 11.0 | 4.1 | 2.0 | 20.0 | 14.0 | 2.0 | — |
| | (D) | Dimethicone (*7) | 0.5 | 0.5 | 0.5 | 0.5 | 0.5 | 0.5 | 0.5 | 0.5 | — |
| | | Tocopherol | 0.1 | 0.1 | 0.1 | 0.1 | 0.1 | 0.1 | 0.1 | 0.1 | 0.1 |
| | | Microcrystalline wax | 1.0 | 1.0 | 1.0 | 1.0 | 1.0 | 1.0 | 1.0 | 1.0 | 1.0 |
| C | (F) | Iron oxide | 2.5 | 2.5 | 2.5 | 2.5 | 2.5 | 2.5 | 2.5 | 2.5 | 2.5 |
| | (B) | PEG-20 tristearate glyceryl (*8) | 2.5 | 2.5 | 2.5 | 2.5 | 2.5 | 2.5 | 2.5 | 2.5 | 2.5 |
| D | | Pentylene glycol | 1.0 | 1.0 | 1.0 | 1.0 | 1.0 | 1.0 | 1.0 | 1.0 | 1.0 |
| | (G) | Water | 2.0 | 2.0 | 2.0 | 2.0 | 2.0 | 2.0 | 2.0 | 2.0 | 2.0 |
| E | (A), (C) | Trimethylsiloxysilicate/ | 30.0 | 20.0 | 15.0 | 10.0 | 35.0 | — | 10.0 | 40.0 | 40.0 |
| | | isododecane = 60/40 (*9) | | | | | | | | | |
| F | | Phenoxyethanol | 0.5 | 0.5 | 0.5 | 0.5 | 0.5 | 0.5 | 0.5 | 0.5 | 0.5 |
| | | Total of (A) component | 23.0 | 17.0 | 14.0 | 11.0 | 26.0 | 5.0 | 11.0 | 29.0 | 29.0 |
| | | Total of (D) component | 4.5 | 10.5 | 13.5 | 6.6 | 4.5 | 22.5 | 16.5 | 4.5 | 0.0 |
| | | (A) + (D) | 27.5 | 27.5 | 27.5 | 17.6 | 30.5 | 27.5 | 27.5 | 33.5 | 29.0 |
| | | (A):(D) | 5.11:1 | 1.62:1 | 1.04:1 | 1.67:1 | 5.78:1 | 0.22:1 | 0.67:1 | 6.44:1 | — |

| | | | | | | | | | | |
|---|---|---|---|---|---|---|---|---|---|---|
| Evaluation | Volume effect | ◯ | ◯ | ◯ | Δ | Δ | Δ | Δ | Δ | Δ |
| | Curling effect | ⊚ | ◯ | ◯ | Δ | ◯ | Δ | Δ | ◯ | ◯ |
| | Water resistance (Normal tempreture) I | ⊚ | ◯ | Δ | ◯ | ⊚ | X | X | ◯ | ◯ |
| | Curl retention effect | ⊚ | ◯ | ◯ | Δ | Δ | Δ | Δ | ◯ | ◯ |
| | Removability by warm water of 40° C. I | ◯ | ◯ | ◯ | ◯ | Δ | ⊚ | ⊚ | X | X |
| | Water resistance (Normal tempreture) II | ⊚ | ◯ | Δ | ◯ | ⊚ | X | X | ⊚ | ⊚ |
| | Removability by warm water (40° C.) II | ◯ | ⊚ | ⊚ | ⊚ | Δ | ⊚ | ⊚ | X | X |

As is apparent from the results shown in Table 1, the mascaras of Examples 1 to 5 are superior in curling effect, water resistance and long-lasting performance on the curling effect and the water resistance as compared with the mascaras of Comparative Examples 1 to 4, and the mascaras of Examples 1 to 5 can be readily removed by warm water alone without using a special remover for a mascara. On the other hand, the mascara of Comparative Example 1 in which only 5% of the oil-soluble film forming agent is contained as the component (A) has unsatisfactory properties as to water resistance, curling effect and long-lasting performance on cosmetic effect. Further, the mascaras of Comparative Examples 3 to 4 are insufficient in removability by warm water. 1: Product name Creasil ID CG (THE INNOVATION COMPANY)2: Product name Rheopearl KL 2 (Chiba Flour Mill Co., Ltd.)3: Product name PINECRYSTAL KE-311 (Harima Chemicals Co., Ltd.)4: Product name Sugar Wax A-10E (DKS Co., Ltd.)5: Product name S Face IS-201P (Sakamoto Yakuhin Kogyo Co., Ltd.)6: Product name T. I. O. (The Nissin Oillio Group, Ltd.)7: Product name Silicone KF-96 A 200 CS (Shin-Etsu Chemical Co., Ltd.)8: Product name EMALEX GWS-320 (Nihon Emulsion Co., Ltd.)9: Product name X-21-5595 (Shin-Etsu Chemical Co., Ltd.)

### Examples 6 to 9 and Comparative Examples 5 to 10

### <Oil-Based Mascara>

Mascaras of the formulation shown in Table 2 were prepared by the same production procedure as described above, and water resistance and removability by warm water were evaluated in accordance with the above evaluation methods f to g. The results are shown in Table 2 together with the formulation.

TABLE 2

| | | | Exampe | | | | | Comparative Example | | | | | |
|---|---|---|---|---|---|---|---|---|---|---|---|---|---|
| Phase | Component | | 6 | 7 | 8 | 9 | 10 | 5 | 6 | 7 | 8 | 9 | 10 |
| A | (C) | Isododecane (*1) | Bal- | Bal- | Bal- | Bal- | Bal- | Bal- | Bal- | Bal- | Bal- | Bal- | Bal- |
| | | | ance | ance | ance | ance | ance | ance | ance | ance | ance | ance | ance |
| | (E) | Dextrin palmitate (*2) | 15.5 | 15.5 | 15.5 | 15.5 | 15.5 | 15.5 | 15.5 | 15.5 | 15.5 | 15.5 | 15.5 |
| B | (A) | Glyceryl hydrogenated rosinate (*3) | 5.0 | 5.0 | 5.0 | 5.0 | 5.0 | 5.0 | 5.0 | 5.0 | 5.0 | 5.0 | 5.0 |
| | (E) | Sucrose acetate stearate (*4) | 1.0 | 1.0 | 1.0 | 1.0 | 1.0 | 1.0 | 1.0 | 1.0 | 1.0 | 1.0 | 1.0 |
| | (D) | Polyglyceryl-2 isostearate (*5) | 2.0 | 2.0 | 2.0 | 2.0 | 2.0 | 2.0 | 2.0 | 2.0 | 2.0 | 2.0 | 2.0 |
| | (D) | Triethylhexanoin (*6) | 2.0 | 8.0 | 11.0 | 20.0 | 2.0 | 14.0 | 2.0 | 2.0 | 2.0 | 2.0 | 2.0 |
| | (D) | Dimethicone (*7) | 0.5 | 0.5 | 0.5 | 0.5 | 0.5 | 0.5 | 0.5 | 0.5 | 0.5 | 0.5 | 0.5 |
| | | Tocopherol | 0.1 | 0.1 | 0.1 | 0.1 | 0.1 | 0.1 | 0.1 | 0.1 | 0.1 | 0.1 | 0.1 |
| | | Microcrystalline wax | 1.0 | 1.0 | 1.0 | 1.0 | 1.0 | 1.0 | 1.0 | 1.0 | 1.0 | 1.0 | 1.0 |
| C | (F) | Iron oxide | 2.5 | 2.5 | 2.5 | 2.5 | 2.5 | 2.5 | 2.5 | 2.5 | 2.5 | 2.5 | 2.5 |
| D | (C) | Isododecane | 3.0 | 3.0 | 3.0 | 3.0 | 3.0 | 3.0 | 3.0 | 3.0 | 3.0 | 3.0 | 3.0 |
| | | Methyl methacrylate crosspolymer | 2.5 | 2.5 | 2.5 | 2.5 | 2.5 | 2.5 | 2.5 | 2.5 | 2.5 | 2.5 | 2.5 |
| E | | Pentylene glycol | 1.0 | 1.0 | 1.0 | 1.0 | 1.0 | 1.0 | 1.0 | 1.0 | 1.0 | 1.0 | 1.0 |
| | (G) | Water | 2.0 | 2.0 | 2.0 | 2.0 | — | 2.0 | 2.0 | 2.0 | 2.0 | 2.0 | 2.0 |
| | (B) | PEG-20 tristearate glyceryl (*8) | 2.5 | — | — | — | 2.5 | — | — | — | — | — | — |
| | (B) | PEG-20 tristearate glyceryl (*10) | — | 2.5 | — | — | — | — | — | — | — | — | — |
| | (B) | Steareth-12 stearate(HLB: 8) (*11) | — | — | 2.5 | — | — | — | — | — | — | — | — |
| | (B) | PEG-6 isostearate (HLB: 9) (*12) | — | — | — | 2.5 | — | — | — | — | — | — | — |
| | (B) | PEG-4 tristearate glyceryl | — | — | — | — | — | 2.5 | — | — | — | — | — |
| | | (HLB: 2) (*13) | | | | | | | | | | | |
| | (B) | PEG-20 hydrogenated | — | — | — | — | — | — | 2.5 | — | — | — | — |
| | | castor oil (HLB: 6) (*14) | | | | | | | | | | | |
| | (B) | Steareth-15(HLB: 12) (*15) | — | — | — | — | — | — | — | 2.5 | — | — | — |
| | (B) | Polysorbate 80 (HLB: 15) (*16) | — | — | — | — | — | — | — | — | 2.5 | — | — |
| | (B) | PEG-40 stearate (HLB: 17.5) (*17) | — | — | — | — | — | — | — | — | — | 2.5 | — |
| F | (A), (C) | Trimethylsiloxysilicate/ | 30.0 | 30.0 | 30.0 | 30.0 | 30.0 | 30.0 | 30.0 | 30.0 | 30.0 | 30.0 | 30.0 |
| | | isododecane = 60/40 | | | | | | | | | | | |
| | | (※9) | | | | | | | | | | | |
| G | | Phenoxyethanol | 0.5 | 0.5 | 0.5 | 0.5 | 0.5 | 0.5 | 0.5 | 0.5 | 0.5 | 0.5 | 0.5 |

| | | | | | | | | | | | | |
|---|---|---|---|---|---|---|---|---|---|---|---|---|
| Evalu- | Water resistance | ⊚ | ⊚ | ⊚ | ⊚ | ⊚ | ⊚ | ⊚ | X | X | X | ⊚ |
| ation | (Normal tempreture) II | | | | | | | | | | | |
| | Removability by warm water | ⊚ | ⊚ | ⊚ | ⊚ | ⊚ | X | X | ⊚ | ⊚ | ⊚ | X |
| | (40° C.) II | | | | | | | | | | | |

(*10): Product name Uniox GT-20IS (NOF Corporation)
(*11): Product name EMALEX-SWS-12 (Nihon Emulsion Co., Ltd.)
(*12): Product name EMALEX PEIS-6 EX (Nihon Emulsion Co., Ltd.)
(*13): Product name EMALEX-GWS-304 (Nihon Emulsion Co., Ltd.)
(*14): Product name EMALEX RWIS-320 (Nihon Emulsion Co., Ltd.)
(*15): Product name EMALEX 615 (Nihon Emulsion Co., Ltd.)
(*16): Product name Rheodol TW-O 120V (Kao Corporation)
(*17): Product name NIKKOL MYS-40V (Nikko Chemicals Co., Ltd.)
(*18): Candelilla resin E-1 (Japan Natural Products Co., Ltd.)

As is apparent from the results shown in Table 2, the mascaras of Examples 6 to 10 were excellent in water resistance and removability by warm water as compared with the mascaras of Comparative Examples 5 to 10. On the other hand, the mascaras of Comparative Examples 5, 6 and 10 containing a nonionic surfactant having an HLB value of 2 or 6 or no nonionic surfactant had insufficient removability by warm water. Further, the mascaras of Comparative Examples 7 to 9 containing a nonionic surfactant having an HLB value of 12 or more had insufficient water resistance.

### Example 10

### <Mascara Makeup Base>

Using the ingredients shown below, a mascara makeup base was prepared according to the following production procedure.

| (Component) | (%) |
|---|---|
| (1) Isododecane (*1) | Balance |
| (2) Dextrin palmitate (*2) | 15.5 |
| (3) Glyceryl hydrogenated rosinate (*3) | 5 |
| (4) Sucrose acetate stearate (*4) | 1 |
| (5) Polyglyceryl-2 isostearate (*5) | 2 |
| (6) Triethylhexanoin (*6) | 2 |
| (7) Dimethicone (*7) | 0.5 |
| (8) Tocopherol | 0.05 |
| (9) Microcrystalline wax | 1 |
| (10) PEG-20 tristearate glyceryl (*8) | 2.5 |
| (11) Pentylene glycol | 1 |
| (12) Water | 2 |
| (13) Trimethylsiloxysilicic acid/isododecane = 6/4 (*9) | 30 |
| (14) Phenoxyethanol | 0.5 |

### (Production Procedure)

A. Components (1) to (14) were heated and homogeneously mixed at about 110° C.
B. The mixed solution obtained in the above A was filled into a container to form a mascara makeup base.

Use of the mascara makeup base obtained as described above prior to application of a commercial mascara was effective to improve curling effect, water resistance and long-lasting performance on cosmetic effect. Further, the mascara makeup base was readily removed together with the mascara by washing with warm water of about 40° C. alone.

### INDUSTRIAL APPLICABILITY

According to the present invention, an oil-based eyelash cosmetic having excellent removability by warm water in addition to excellent curling effect and long-lasting performance of cosmetic effect such as water resistance peculiar to the conventional oil-based eyelash cosmetics is provided. Further, when using this oil-based eyelash cosmetic, makeup can be readily removed by washing with warm water without using a special remover. Therefore, removal of makeup can be efficiently performed.

1. An oil-based eyelash cosmetic comprising, 3 to 40% by mass of an oil-soluble film forming agent (A),
0.1 to 10% by mass of a nonionic surfactant having an HLB value of 7 to 10 (B),
20 to 70% by mass of a volatile oil (C) and
0.5 to 30% by mass of a nonvolatile oil (D),
wherein the ratio by mass of the component (A) to the component (D) [(A)/(D)] is 0.7/1 to 5.8/1, and when a solid oil having a melting point of 50° C. or higher is contained, its content is 10% by mass or less.

2. The oil-based eyelash cosmetic according to claim 1, further comprising 0.5 to 40% by mass of an oil-based gelling agent (E).

3. The oil-based eyelash cosmetic according to claim 1, further comprising 0.1 to 20% by mass of a coloring material (F).

4. The oil-based eyelash cosmetic according to claim 1, further comprising 5% or less by mass of water (G).

5. The oil-based eyelash cosmetic according to claim 1, wherein the content of the solid oil having a melting point of 50° C. or higher is 5% by mass or less.

6. The oil-based eyelash cosmetic according to claim 1, wherein when a nonionic surfactant having an HLB value exceeding 10, an anionic surfactant, a cationic surfactant or an amphoteric surfactant is contained, the content thereof is 0.5% by mass or less.

7. The oil-based eyelash cosmetic according to claim 1, wherein the component (A) is trimethylsiloxysilicate, rosin-based resin or candelilla resin.

8. The oil-based eyelash cosmetic according to claim 1, wherein the component (B) is polyoxyethylene fatty acid ester, polyoxyethylene fatty acid glyceryl or fatty acid polyoxyethylene alkyl ether.

9. The oil-based eyelash cosmetic according to claim 2, wherein the component (E) is dextrin fatty acid ester, inulin fatty acid ester, sucrose fatty acid ester, starch fatty acid ester, organically modified clay mineral, metal soap or 12-hydroxystearic acid.

10. The oil-based eyelash cosmetic according to claim 1, wherein it is a mascara or a mascara makeup base.

11. A cosmetic method for making up eyelashes comprising makeup of the eyelashes using the oil-based eyelash cosmetic according to claim 1, and removal of the makeup by cleansing with warm water without using a remover.
